(19) United States
(12) Patent Application Publication
(10) Pub. No.: US 2011/0105826 A1
(43) Pub. Date: May 5, 2011

(54) TRANSCRANIAL MAGNETIC STIMULATION BY ENHANCED MAGNETIC FIELD PERTURBATIONS

(75) Inventors: David J. Mishelevich, Playa del Ray, CA (US); M. Bret Schneider, Portola Valley, CA (US)

(73) Assignee: NEOSTIM, INC., Redwood City, CA (US)

(21) Appl. No.: 12/990,235

(22) PCT Filed: May 26, 2009
(86) PCT No.: PCT/US2009/045109

§ 371 Date: Nov. 30, 2010

Related U.S. Application Data
(60) Provisional application No. 61/055,463, filed on May 23, 2008.

(51) Int. Cl. A61N 2/02; A61N 2/00

(52) U.S. Cl. 600/13; 600/9

(57) ABSTRACT

Described herein are devices, systems and methods to enhance the magnetic perturbation of a neuronal (e.g., brain) target during Transcranial Magnetic Stimulation (TMS), thereby enhancing the induced current in the target. In general, these devices, systems and methods enhance the magnetic perturbation (dB/dt) of the target by mechanically moving a TMS electromagnet (e.g. coil) at a frequency of greater than 1 kHz.

### CROSS REFERENCE TO RELATED APPLICATIONS

This patent claims priority to U.S. Provisional Patent Application Ser. No. 61/055,463, filed on May 23, 2008 and titled “PHYSICAL COIL MOVEMENT FOR ENHANCED MAGNETIC FIELD PERTURBATIONS”.

This application may be related to U.S. Pat. No. 7,520,848 to Schneider et al.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices and methods described herein relate generally to the control of Transcranial Magnetic Stimulation (TMS). In particular, described herein are methods, devices and systems for TMS by precisely moving the TMS electromagnet(s) to enhance perturbations of neural tissue.

### BACKGROUND OF THE INVENTION

Transcranial magnetic stimulation (TMS) is a noninvasive technique to stimulate neurons in the brain. TMS induces a weak electric current in neuronal tissue by applying a rapidly changing magnetic field, resulting in electromagnetic induction. In theory, TMS may evoke a response from neurons in the brain by inducing current at or close to the axon hillock, resulting in stimulation of the nerve. Nerve fibers that are parallel to the TMS coil (e.g., perpendicular to the applied magnetic field) are believed to be more likely to depolarize than those perpendicular to the coil. Thus, bending nerve fibers may be more susceptible to TMS effects than straight fibers.

Typically, TMS is applied by application of a TMS electromagnet to the outside of the subject's head. For example, a coil of wire, encased in plastic, is held to the head. When the TMS electromagnet (coil) is energized by the rapid discharge of a large capacitor, a rapidly changing current flows in its windings. The rapidly changing current produces a magnetic field oriented orthogonally to the plane of the coil. The magnetic field passes unimpeded through the skin and skull, inducing an oppositely directed current in the brain that flows tangentially with respect to skull. The current induced activates nearby nerve cells. Because of the irregular shape of the brain, and the often tortuous pathways of various neurons in the brain, the resulting current path is complex and may be difficult to predict or model. Thus it is often difficult to estimate the efficiency of stimulation for any given position and power of the TMS electromagnet.

Repetitive TMS (rTMS) may be used to treat specific neurological and psychiatric illnesses, and may require that specific neuroanatomical structures be targeted using specific pulse parameters. The strength and frequency of the stimulation applied may be tailored to the target neuronal structure and the desired effect. For example, rTMS frequencies of around 1 Hz have been shown to produce inhibition of motor cortex, while higher-frequency stimulation may produce facilitation. In addition, the strength of the applied magnetic field must be sufficient to stimulate the target structure.

Effective TMS depends on perturbing the electrical environment at the neural target to stimulate neural activity. Current is induced in the neuron by the varying applied magnetic field. Typically, the magnetic field is varied by pulsing the magnetic field. Described herein are methods of varying the applied magnetic filed by moving the TMS electromagnet. Appropriate movement (e.g., perturbing motion, as described below), may supplement the pulsing of the magnetic field, and may enhance the current induced in the neuron in previously unavailable ways. As described herein, the frequency and direction of the perturbing motion may therefore be configured to enhance the evoked current.

Many of the TMS systems and configurations previously described include of one or more TMS electromagnets having static or unmoving coils. Although movement of TMS electromagnets has been described, not all movement of a TMS electromagnet will result in enhancement of the induced current. Unlike gross movements of the TMS electromagnet around the head, or movement between stimulation of target regions, the perturbing movement described herein typically results in an enhanced induced current and stimulation of a neuronal target.

For example, Schneider and Mishelevich, in U.S. Pat. No. 7,520,848, describe moving TMS electromagnets around the head of the patient to avoid overstimulation of superficial tissues by applying the magnetic field through different trajectories such that the target is consistently stimulated but the overlying tissues. However, in this example, field perturbations within each single pulse are typically limited to the energy and the time course of the signal produced by the pulse generation unit (PGU) output. The gross movement of the coils around the head are not perturbing motion, and does not vary the applied magnetic field on the same time scale as the pulse generation unit.

Devices, systems and methods for mechanically perturbing the TMS electromagnet to modify the applied magnetic field would be useful for enhancing the control and level of stimulation applied during TMS. It would be desirable to have a method for further perturbing and thereby shaping the magnetic field applied to a neuron. The devices, systems and methods described herein are directed to the application of perturbing motion to change the magnetic field per unit time (dB/dt) seen by a neuron or portion of a neuron. Such perturbations are applicable whether a grossly moving or relatively static electromagnet or array of electromagnets is involved. These devices and systems may allow one or more TMS electromagnets to more effectively stimulate neurons by applying a time-varying magnetic field over more neuronal area, and in more directions along the one or more neurons, thereby potentially increasing the likelihood of evoking a current in the neurons. This may allow the application of electromagnetic stimulation at lower powers.

Thus, the present invention provides devices for physically moving (in perturbing motion) one or more pulsed electromagnets to achieve rapid perturbation of the stimulating magnetic field. This may alter the change in magnetic field per unit time (dB/dt) and therefore produce more effective electrical current induction at the target site, e.g., a cluster of neurons, within a subject's brain.

### SUMMARY OF THE INVENTION

Described herein are devices, systems and methods for Transcranial Magnetic Stimulation involving the use of one or more magnetic coils, pulsed together or serially. The magnetic coils are moved in a perturbing motion in the x, y, and/or z motion, so that the magnetic field emitted by the TMS electromagnet and the consequently stimulated electrical fields are changed quickly enough to allow perturbation of the local electrical environment at the target to facilitate local stimulation. The enhanced changing magnetic field (dB/dt) resulting from the perturbing motion many enhance the effect of the magnetic field on the target neural structure(s). This effect of the perturbing motion of the TMS electromagnet can be achieved with any TMS electromagnet, irrespective of coil shape, type or higher-order configuration within an array of electromagnets, as long as they can physically be moved in a manner consistent with this invention.

As used herein, perturbing motion of one or more TMS electromagnet typically refers to the motion of the TMS electromagnet in one or more of X, Y, and Z, along the axis of the TMS electromagnet (or electromagnets). The perturbing motion may be an oscillatory (e.g., rotational) motion in one or more of Z, Y and Z. In particular, the oscillatory motion may be oscillation (including two- or three-dimensional oscillatory motion) at a frequency within the range of the pulsing frequency of a typical static TMS electromagnet (e.g., the frequency of the pulsing generation unit). For example, the frequency of oscillatory movement may be greater than about 1 kHz, including about 2 kHz to about 20 kHz, about 2 kHz to about 10 kHz, or any sub-range thereof. This frequency may result in perturbation of the magnetic field at the neuronal target in approximately the same scale as the electrical pulsing. Thus, in some variations, the electrical pulsing may be decreased (or eliminated) in favor of the applied perturbing motion. Generally, however, the perturbing motion is applied in conjunction with pulsing of the TMS electromagnet.

Stimulation using a TMS electromagnet that is moved in a perturbing motion may therefore reflect the magnitudes of the X, Y, and Z directional components of the magnetic field emitted by the TMS electromagnet, and not simply the overall power. As mentioned, the devices and systems for perturbation of the axial magnitudes may move the TMS electromagnet by rotating one or more TMS electromagnets around their central axes, by advancing and withdrawing the electromagnets along those central axes (e.g., Z axis), and/or displacing the electromagnets lateral to and from the central axes (X or Y axes), or by some combination thereof. The actual movement of the TMS electromagnet(s) may be accomplished by one or more TMS perturbing actuators, and the movements may be relatively small at the TMS electromagnets. For example, movement of a TMS electromagnet may result in a small movement seen by the target axons within the mm range (e.g., between 0.1 and 9 mm, etc.) of the emitted field.

For example, described herein are TMS systems that include a TMS electromagnet, a perturbing actuator connected to the TMS electromagnet and configured to mechanically oscillate and/or rotate the TMS electromagnet at a frequency of greater than 1 kHz, and a controller configured to trigger activation of the TMS electromagnet and mechanical oscillation (e.g., rotation) of the TMS electromagnet.

In some variations the TMS systems also include a support configured to hold the TMS electromagnet in position relative to a subject's head. For example, the support may be a gantry, a framework, a helmet, or the like.

The perturbing actuator may be a driver such as a voice coil, a piezoelectric actuator, or the like. The perturbing actuator typically oscillates the TMS electromagnet relative to the central axis of the TMS electromagnet. As used herein, oscillation or oscillatory motion included rotational motion or movement. The central axis of the TMS electromagnet may be the axis of the direction of the emitted magnetic field from the TMS electromagnet. Thus, the perturbing actuator may be configured to rotate the TMS electromagnet on its central axis. In some variations, the perturbing actuator is configured to move the TMS electromagnet in and out on its central axis. In some variations, the perturbing actuator is configured to move the TMS electromagnet laterally on and off the central axis. In other variations, the perturbing actuator is configured to move the TMS electromagnet laterally partially on and off the central axis by moving the TMS electromagnet about a fixed point on the central axis. Any combination of these movements may be used.

The TMS electromagnet may be movably fixed to a support. For example, one portion of the TMS electromagnet may be pivotally fixed (e.g., to a frame) so that the perturbing actuator applies force to move the TMS electromagnet relative to the support. A pushing/pulling or backward/forward (or other vibratory motion) by the actuator may pivot the TMS electromagnet in the support so that the emitted field is oscillated. In some variations a return element (including a spring or other restoring element) applies a return force in conjunction with the actuator to oscillate the TMS electromagnet.

In some variations the TMS system may also include a housing enclosing the TMS electromagnet. The housing may also include attachment sites for the actuator and/or the support.

The TMS systems described herein may also include a plurality of TMS electromagnets. In some variations the plurality of TMS electromagnets may all be oscillated or driven by the same perturbing actuator. In other variations all (or a subset) of the TMS electromagnets may be oscillate or driven by separate perturbing actuators. For example, the devices described herein may include a second TMS electromagnet and a second perturbing actuator connected to the second TMS electromagnet and configured to mechanically oscillate the second TMS electromagnet at a frequency of greater than 1 kHz.

The perturbing motions performed by the perturbing actuators described herein are generally “small” movements of the TMS electromagnets, as compared to the gross movements of the TMS electromagnets around or about the subject's head. For example, the oscillatory movement of the TMS electromagnet caused by the perturbing actuator may move the TMS electromagnet on the order of millimeters (e.g., less than 10 mm). The motion is also oscillatory, meaning that it moves in a repeated path (which does not have to be exactly the same with each pass), typically centered around a central position. The movements are typically quite rapid compared to other movements of the TMS electromagnets around or about the subject's head. It should be understood that the TMS electromagnets may be moved both in the oscillatory motion of a perturbing actuator as described herein as well as in a gross movement around the subject's head. For example, a TMS system may be configured for both perturbing oscillatory motion (as described herein) at greater than 1 kHz, as well as gross movements such as movements about the subject's head (as described in U.S. Pat. No. 7,520,848 and WO 2009/033150, for example), or other slower movements.

The TMS systems described herein may also include a coupling shaft connecting the perturbing actuator to the TMS electromagnet. In some variations, the coupling shaft may be configured to further isolate the actuator from the relatively high magnetic field associated with the TMS electromagnet. For example, the coupling shaft may space the actuator (e.g., a voice-coil or other magnetic-based actuator) from the TMS electromagnet. The coupling shaft may be a non-magnetizable (non-ferromagnetic) material.

The TMS system may be configured so that the perturbing actuator mechanically oscillates the TMS electromagnet at a frequency of between about 1 kHz and about 20 kHz, or about 2 kHz and about 10 kHz or about 1 kHz and about 10 kHz, or any range between 1 kHz and 20 kHz.

For example, described herein are TMS systems comprising: a TMS electromagnet; a perturbing actuator connected to the TMS electromagnet comprising a voice coil, wherein the perturbing actuator is configured to mechanically oscillate the TMS electromagnet at a frequency of between about 1 kHz and 10 kHz; and a controller configured to trigger activation of the TMS electromagnet and mechanical oscillation of the TMS electromagnet.

Also described herein are methods of applying Transcranial Magnetic Stimulation (TMS) to a subject that include the steps of: positioning a TMS electromagnet toward a target brain region; emitting a magnetic field from the TMS electromagnet towards the target tissue; and mechanically oscillating the TMS electromagnet at a frequency of greater than 1 KHz so that magnetic field emitted by the TMS electromagnet moves relative to the target tissue and electrically perturbs the target tissue.

The mechanical oscillation of the TMS electrode may be coordinated with the emission of the magnetic field from the TMS electromagnet. For example, the TMS electromagnet may be oscillated before the activation of the electromagnet, or the two may be synchronously operated, so that the triggering of the TMS electromagnet also triggers mechanical oscillation the TMS electromagnet.

The step of positioning the TMS electromagnet may include fixing the position of the TMS electromagnet relative to a subject's head. For example, the TMS electromagnet (or electromagnets in variations including more than one) may be held in a relatively fixed position with respect to the subject's head while the TMS electromagnet is oscillated.

As mentioned above, the step of mechanically oscillating the TMS electromagnet may comprise rotating the TMS electromagnet on its central axis, moving the TMS electromagnet in and out on its central axis, moving the TMS electromagnet laterally on and off the central axis, moving the TMS electromagnet laterally partially on and off the central axis by moving the TMS electromagnet about a fixed point on the central axis, or any combination of these movements.

The step of mechanically oscillating the TMS electromagnet may include oscillating the TMS electromagnet at a frequency of between about 1 KHz and about 20 kHz (e.g., 2 kHz and 10 kHz, etc.).

### BRIEF DESCRIPTION OF THE DRAWINGS

FIG. 1A illustrates an exemplary TMS electromagnet emitting a magnetic field towards a neuronal target. FIGS. 1B to 1D illustrate various oscillatory movements of the TMS electromagnet described in FIG. 1A.

FIG. 2A schematically illustrates one variation of a system including a TMS electromagnet and a perturbation actuator configured for rotational perturbation of the electromagnet around the central axis of the field emitted by the TMS electromagnet towards the target.

FIGS. 2B and 2C schematically illustrate variations of systems including a TMS electromagnet and a perturbation actuator configured to move the TMS electromagnet in and out along the central axis of the field emitted by the TMS electromagnet towards the target.

FIG. 3 schematically illustrates another variation of a system including a TMS electromagnet and a perturbation actuator configured to move the TMS electromagnet back and forth perpendicular to the central axis of the field emitted by the TMS electromagnet towards the target.

FIG. 4A illustrates one variation of a double coil on a central axis of rotation, with degrees of rotation marked, and an off-center sample region of interest marked with an “X.”

FIG. 4B illustrates the magnetic field strength received at a point beneath the marked region in FIG. 4A as a function of rotation or oscillation of the coil.

FIG. 5A illustrates a double-coil design having square turns, which may create abrupt changes in magnetic field as the coil is oscillated (e.g., rotated on its face).

FIG. 5B Illustrates a double coil design with rounded turns and posteriorly-deflected lateral margins.

### DETAILED DESCRIPTION OF THE INVENTION

The devices, systems and methods described herein are intended to enhance the magnetic perturbation of a neuronal (e.g., brain) target during Transcranial Magnetic Stimulation (TMS), thereby enhancing the induced current in the target. In general, these devices, systems and methods enhance the magnetic perturbation (e.g., dB/dt) of the target by mechanically moving a TMS electromagnet (e.g., coil) at a frequency of greater than 1 kHz.

The principles of the present invention do not depend on the coil shape, type or higher-order configuration within an array of electromagnets, as long as electromagnetic field generated has a shaped component that is directed towards the target. Accordingly, the figures and illustrations described below depict generic electromagnet shapes, though it is to be understood that any appropriate configuration may be used. One example of a TMS electromagnet configuration is a figure-eight, double coil, including those sold by Medtronic (e.g., MC-B70 double coil) and Magstim (e.g., 70 mm double coil). In variations in which more than one coil or coil array is included, the coils may of the same type or a combination of types. Also described herein are coil configurations and shapes that may be of particular interest for use with the perturbing actuators described herein, because oscillation of these configurations (e.g., rotation) results in enhanced dB/dt at a target.

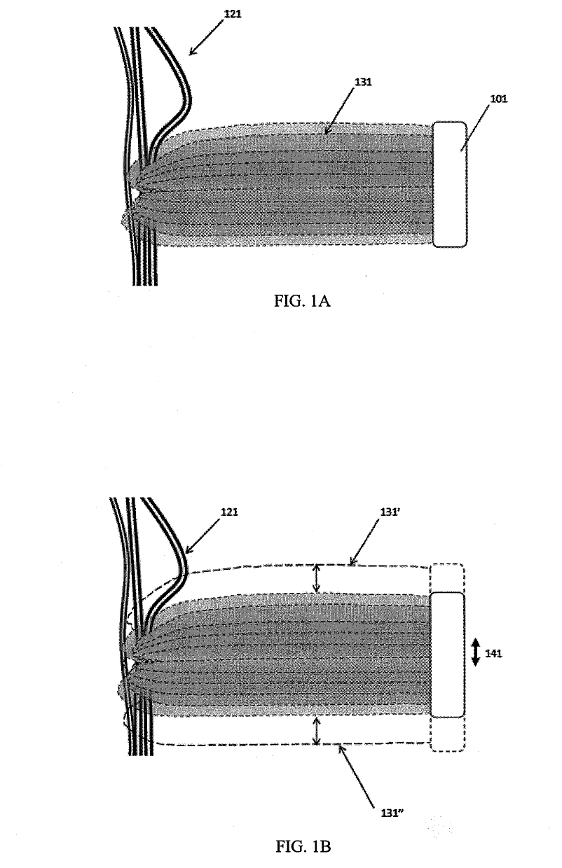

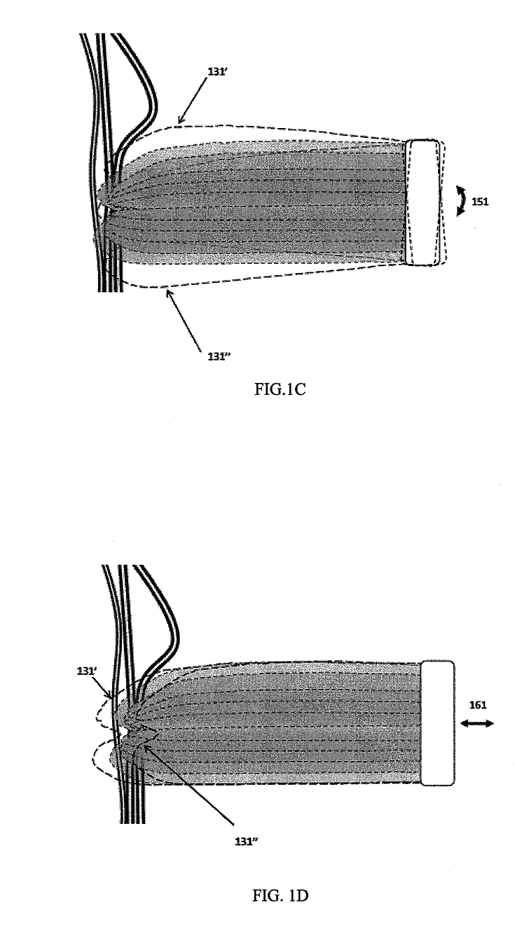

FIGS. 1A-1D illustrates the effect of oscillatory movement of a TMS electromagnet 101 to enhance dB/dt (changes in the magnetic field) seen at a neuronal target (fiber bundle 121). In this simplified illustration, the neuronal target is a bundle of fibers 121 that may include partial or entire neurons in a target brain region. The paths of some of the neuronal fibers 121 extend at different orientations. For the sake of clarity, this simplified model the intervening tissues (e.g., non-target tissues including the scalp, skull and other brain regions) have been omitted. In FIG. 1A, the TMS electromagnet 101 is shown emitting a magnetic field towards the target tissue, as illustrated by the shaded gradient lines 131. In FIG. 1A the TMS is not oscillated, and may be mechanically “static” (although the emitted field may be pulsed at some frequency). Thus, the neuronal fibers in the target brain region 121 are exposed to the varying magnetic field emitted by the (non-moving) TMS electromagnet. The emitted field (shown by the flux lines 131) may vary based on the action of a pulse generation unit (PGU) associated with the TMS electromagnet. The PGU may trigger stimulation of the TMS electromagnet at a high-frequency (often biphasic) signal that will result in a rapidly time-varying magnetic field 131 directed to the target tissue. This results in a change in the magnetic field seen at the target 121, dB/dt, and therefore an induced current.

FIGS. 1B-1D illustrate different variations of oscillation (movement) of the TMS electromagnet illustrated in FIG. 1A. In FIG. 1B the TMS electromagnet is physically oscillated laterally, perpendicular to the central axis of the emitted field from the TMS electromagnet, as indicated by the arrow 141. As a result, the emitted field 131 seen by the target tissue is moving; the magnetic field reaching the nerve bundles 121 (target) is moving across the target region shown. The dashed lines 131′, 131″ illustrate the extent of the movement across the target region. Thus, as the TMS electromagnet is moved laterally, the emitted field is moves relative to the target tissue, further enhancing the change in the magnetic field seen at the target (dB/dt). The resulting change, effecting the induced current, may be reflected by the intensity of the emitted field seen by the target tissue, as well as the rate of movement of the TMS coil (typically greater than 1 kHz) and the rate of change of the emitted field (e.g., due to the pulsing produced by the pulse generation unit output). The lateral movement shown in FIG. 1B may also be referred to in terms of coordinates such as x, y, and z (or other coordinate systems), relative to the magnetic field. For example, in FIG. 1B the filed may be oscillated in the x direction, the y, direction, or any combination of x and y (including oscillating in the xy plane in a circle, ellipse, or any other shape).

In addition to changing the dB/dt seen by all of a portion of the target tissue, physical movement of the magnetic field as shown in FIGS. 1B-1D may also enhance the TMS by applying stimulation to a slightly larger region of the target, as illustrated. In some variations, the direction of the applied magnetic field seen by different regions of the target may also be varied by the perturbing motion of the TMS electromagnet. For example, in FIG. 1B, movement of the TMS electromagnet from the middle position to the upper position translates the changing magnetic field over a region of the target nerve bundle that is bent 121.

FIG. 1C illustrates another variation of an oscillatory motion 151 on a TMS coil. In FIG. 1C the TMS electromagnet of FIG. 1A is oscillated by moving the TMS electromagnet about a fixed point on the central axis, effectively tilting 151 the TMS electromagnet as shown. In this variation, the emitted field 131′, 131″ is moved along the target tissue at the rate of oscillation, as just described. The tilting motion may result in a direction of change of the applied magnetic field seen at the target tissue in all of x, y and z.

FIG. 1D illustrates a third variation of oscillatory motion, in which the TMS electromagnet is moved back and forth 161 along the central axis of the emitted magnetic field of the TMS electromagnet. Relative to the target tissue, the applied electromagnet field is moving in the z direction (changing the ‘depth’).

Any of the method of movement described herein (including in the figures and example described below) may be combined, so that the magnetic field emitted by the TMS electromagnet (a plurality of TMS electromagnets) may be oscillated in any appropriate two dimensional or three dimensional pattern relative to the target tissue.

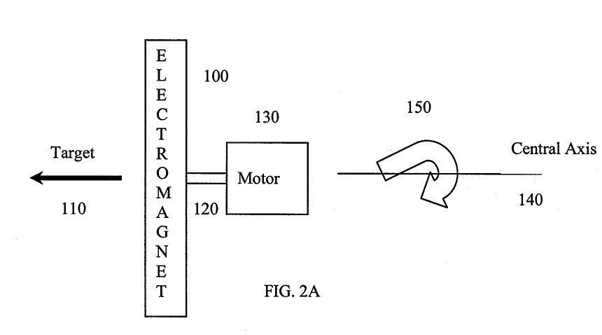

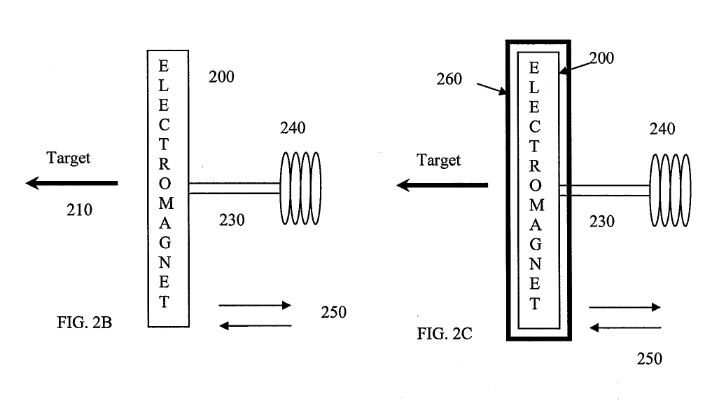

As mentioned, rapid movements of one or more electromagnets in appropriate directions may allow perturbations of the local electrical fields at the target neural structures. For example, in FIG. 2A, TMS electromagnet 100 is aimed toward the target (direction shown by arrow 110) and turned by shaft 120 by motor 130 so the electromagnet 100 is rotated around central axis 140 as indicated by arrow 150. This provides for a rotational perturbation. As a result of this rotation, the change in the strength of the field striking the target, as a function of time (dB/dt) is altered in proportion with not only the rise and the fall of the electrical pulse traveling through the coil, but also with the rotational speed of the coil. In any of these variations, the TMS electromagnet(s) may be driven by one or more perturbing actuators, which may be coupled directly or indirectly to the TMS electromagnets. The perturbing actuator is typically an actuator (e.g., a voice coil actuator, a piezoelectric actuator, etc.) that provides force to oscillate the TMS electromagnet. The actuator may be coupled to a connector or support that couples the TMS electromagnet to the motion of the actuator. In some variations, the actuator motion is translated into the oscillatory motion at the TMS electromagnet by a coupling on the TMS electromagnet or a frame or housing for the TMS electromagnet. For example, the TMS coil may be coupled to a hinge or joint that allows it to pivot as it is driven by the actuator. In some variation a restoring force may be applied (e.g., from a spring or the like), or a mechanical dampener or the like may also be used (or designed into the material properties of the couplings or holders) to control the oscillation of the TMS electromagnet.

In FIG. 2B, electromagnet 200 is aimed toward the target (direction shown by arrow 210) and moved in and out along the central axis of the electromagnet (as indicated by bi-directional arrows 250) by shaft 230 driven by speaker coil 240. Speaker coil 240 is composed of a combination of a speaker-coil permanent magnet and a concentrically located speaker-coil electromagnet in which the current in the speaker-coil electromagnet is rapidly reversed and thus the speaker-coil electromagnet is alternately attracted and repelled by the speaker-coil permanent magnet. This causes rapid back and forth movement of the speaker-coil electromagnet and what is attached to it. In the example, the speaker-coil electromagnet is attached to the Transcranial Magnet Stimulation electromagnet and moves that electromagnet in and out along its central access. This provides for a linear perturbation along the central axis. FIG. 2C shows a similar arrangement with the exception that electromagnet 200 is enclosed in cover 260. Note that any of the electromagnets shown in the figures or otherwise covered could be so enclosed. Power may be delivered to the coils via a slip ring which maintains continuity of the positive and negative poles of the power supply throughout rapid movements. Once such high-power slip ring is made by a division of Northrop (Blacksburg, Va.).

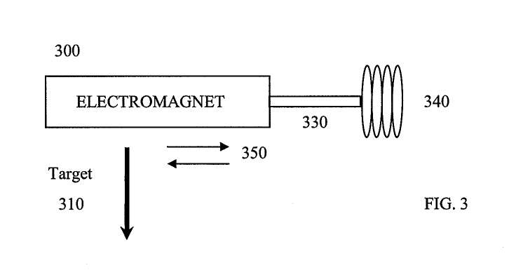

In the example shown in FIG. 3, electromagnet 300 is aimed toward the target (direction shown by arrow 310) and moved laterally (perpendicular to the central axis) back and forth (as indicated by bi-directional arrows 350) by shaft 330 driven by speaker coil 340. This provides for a linear perturbation perpendicular to the central axis, similar to the example shown in FIG. 1B.

As mentioned, other oscillating motions the TMS electromagnet are possible. For example, the electromagnets can be rotated around a point on the central axis. While only electromagnet is shown in each case, fixed arrays, or a set or two more independent electromagnets are handled in the same way. Also, one or more of the motions covered in FIGS. 1B-3, and/or alternative motions can be combined.

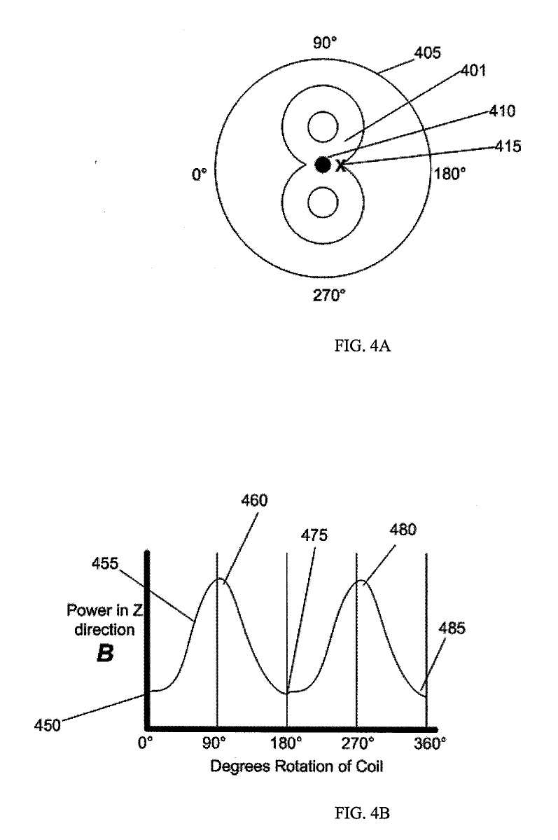

As used herein oscillation includes rotation of the TMS electromagnet, and may also include rotation in only one direction. In general, the “oscillation” of the TMS electromagnet refers to the movement of the emitted magnetic field at target tissue. Even rotation in a single direction (e.g., clockwise) will result in an oscillation of the emitted field at the target, thereby changing dB/dt, as illustrate in FIGS. 4A and 4B. For example, FIG. 4A illustrates a double coil 401 on an axis of rotation 410, with degrees of rotation marked (external to outer circle 405). Off-center sample region of interest 415 marked with an “X”.

FIG. 4B illustrates the magnetic field strength received at region of interest 415 from FIG. 4A, shown as a function of rotation of coil 401 from FIG. 4A. For the purposes of this exemplary illustration (shown in FIG. 4B), the power passed through coil 401 may be presumed to be held at a steady level for the period of time during which the rotation of the coil takes place. In actuality, as mentioned above for the typical TMS system, the power through the coil generally passes surges in a rapid (approx 0.1 ms) pulse. This rises as a function of time during the rotation. However, this steady-state assumption may be approximated if the rotation of the coil is very fast (less than, for example 0.1 ms per rotation), or if the magnet holds a steady flux. This latter case would permit strong permanent magnets to be used for magnetic stimulation in the context of the present invention, since a dB/dt would result from the physical movement of the magnet past a neuron. Alternatively, a steady-state electromagnet (for example those used in the main solenoid of an MRI scanner) may also be used to create stimulation through physical movement. At 0° rotation 450, the power level is low. This rises over the time of a quarter-rotation (455) to a high point at 90°, back to the low value at 180°, up to the high value again at 270° and to its original low level again at 360°.

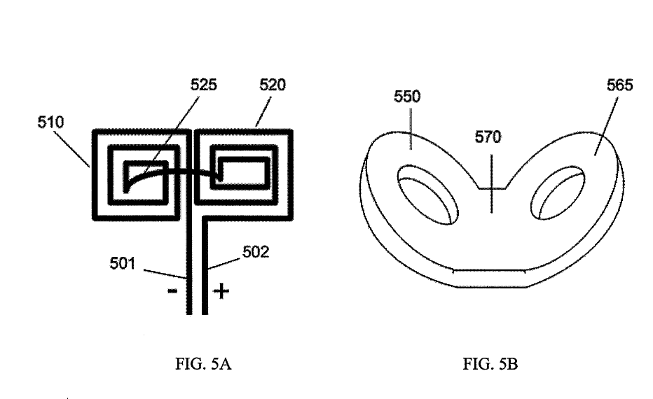

As mentioned, any appropriate TMS electromagnet may be used. It may also be possible to optimize or match the oscillatory motion of the TMS electromagnet with the design of the TMS electromagnet. For example, FIG. 5A illustrates a double-coil design having square turns, which may create abrupt changes in magnetic field as the coil is rotated on its face (e.g. oscillation as illustrated in FIG. 2A. Positive lead 502 and negative lead 501 connect to one another by a series of sharp (e.g. 90°) bends of a double (component coil 510 and component coil 520) concentric coil with currents passing in opposite directions. A bridge between the two coils is provided by conductive segment 525. Such a coil may be constructed using materials including copper flat wire insulated with Kapton™.

FIG. 5B illustrates the smooth insulated shell of a double coil design, this one with rounded turns and posteriorly-deflected lateral margins 550 and 565. Center 570 of this double coil remains closest to the target beneath.

Perturbations induced by mechanically oscillating the TMS electromagnet as described herein may be used with any firing pattern of the TMS electromagnet. Where there are two or more electromagnets or arrays, they are applicable whether the electromagnets or arrays are fired sequentially or simultaneously. In another embodiment, the movements of the electromagnet or electromagnets are phase locked with the stimulating pulses.

As discussed above, the change in magnetic field (dB/dt) induces electrical current within neurons, including neurons of the target tissue. The dB/dt may be below a physiologically threshold, or it may be brought to a physiologically effective level. For example, the current induced by the changing magnetic field may trigger an action potential in the target neuron(s). dB/dt may be controlled for nerve stimulation by (1) controlling the speed of the emitted magnetic pulse (e.g., its onset and its dissipation) emitted by TMS electromagnet, and (2) controlling the travel speed (physical movement) of the TMS electromagnet(s) that is/are the magnetic field source.

Although the discussion above focuses predominantly on the control of the travel speed of the TMS electromagnet, control of the travel speed may be combined or informed by the control of the rate of emitted magnetic field (e.g., pulsing), including control of the energy applied to activate the TMS electromagnet. For example, if a pulsed magnetic field is used, quickening the pulse duration at a given power level to, say a 0.01 ms duration, the travel speed of the moving magnet need only be 1/10 as fast is it needs to be at the same power level with the pulse occupying a 0.1 ms time period. By this same principal, a static magnet moved at a 1 kHz rate may be sufficient to produce depolarization at an adjacent neuron. Taking this principal yet further, if the magnet is physically moved at a yet faster speed, say 2 kHz, then a static-field magnet of ½ the strength of the previous case may produce the same level of current induction (and hence stimulation) within the targeted nerve. Thus, any target dB/dt may be determined by combining the effect of the rate of movement and the rate of pulsing of the TMS electromagnet.

A wide range of magnet movement rates may be used. The rate may be determined in part by the rate of pulsing of the applied magnetic field, which may be determined by the rate of applied energy to stimulate the TMS electromagnet(s). For example, the rate of oscillation of the TMS electromagnet(s) may depend upon the magnitude and direction of the magnetic field pulse emitted. The methods and devices described herein may achieve dB/dt values comparable to those of standard TMS systems (e.g., “static” TMS systems), in which the coil is entirely stationary, and a high power (2 Tesla) and short duration (less than 0.1 ms) filed is emitted. Using the methods and devices described herein, a lower power level may be used with longer pulse durations, by agitating (e.g., oscillating) the TMS electromagnet at faster movement rates. For static (e.g., steady-state) and weaker magnetic fields, the oscillation rate of the moving magnet may be faster to achieve comparable dB/dt values: e.g., the TMS electromagnet(s) may be oscillated up to 10 kHz. For pulsed magnetic field, particularly stronger pulsed fields, very slow perturbation movements may be required to achieve comparable stimulation.

In some variations, the system may be tuned to one or both of the characteristics of the (a) physical motion imposed on the electromagnet or electromagnets, and (b) phase-lock relationship between the stimulating pulses and the physical motion, if phase locking is used. Tuning may be accomplished by means including performing the Transcranial Magnetic Stimulation while simultaneously acquiring PET imaging, preferably using labeled oxygen or water to tune by obtaining feedback and seeing the impact of changes and phase locking (if phase locking is used). Control can either be accomplished manually or by using automatic feedback based on a selected characteristic of the image.

The methods and devices described herein may be used or adapted for use with TMS systems including TMS electromagnets that are either static or kept in a relatively fixed position relative to the subject's head during stimulation, or in TMS systems in which one or more of the TMS electromagnets are moved relative to the subject's head. As mentioned above, U.S. Pat. No. 7,520,848 to Schneider et al. describes systems in which deep-brain TMS may be achieved by moving the TMS electromagnet(s) relative to the brain target around the subject's head; moving them in this manner may allow summation of the TMS electromagnetic field at the target brain region, while avoiding over-stimulation of non-target intervening regions that may be located more superficially between the target tissue and the TMS electromagnet(s). The methods and devices described herein may be used with TMS electromagnets that are moved during stimulation (including system that move the TMS electromagnets at less than 1 kHz).

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. For instance, such changes may include variations in the amplitude or frequency of the stimulation. Such modifications and changes do not depart from the true spirit and scope of the present invention, which is set forth in the following claims.

1. A Transcranial Magnetic Stimulation system, the system comprising:
a magnet configured to be positioned around a subject's head;
a perturbing actuator connected to the magnet and configured to mechanically oscillate the magnet at a frequency of greater than 1 kHz; and
a controller configured to trigger activation of the magnet and mechanical oscillation of the magnet to delivery transcranial magnetic Stimulation to the subject.

2. The device of claim 1 further comprising a support configured to hold the magnet in position relative to a subject's head.

3. The device of claim 1, wherein the perturbing actuator is configured to rotate the magnet on its central axis.

4. The device of claim 1, wherein the perturbing actuator is configured to move the magnet in and out on its central axis.

5. The device of claim 1, wherein the perturbing actuator is configured to move the magnet laterally on and off the central axis.

6. The device of claim 1, wherein the perturbing actuator is configured to move the magnet laterally partially on and off the central axis by moving the magnet about a fixed point on the central axis.

7. The device of claim 1 further comprising a housing enclosing the magnet.

8. The device of claim 1 further comprising a second magnet and a second perturbing actuator connected to the second magnet and configured to mechanically oscillate the second magnet at a frequency of greater than 1 kHz.

9. The device of claim 1 wherein the magnet is part of an array of magnet and the perturbing actuator is configured to mechanically oscillate the array of magnet.

10. The device of claim 1, wherein the perturbing actuator comprises a voice coil.

11. The device of claim 1, wherein the perturbing actuator comprises a piezoelectric actuator.

12. The device of claim 1 further comprising a coupling shaft connecting the perturbing actuator to the magnet.

13. The device of claim 1, wherein the perturbing actuator is configured to mechanically oscillate the magnet at a frequency of between about 2 kHz and about 10 kHz.

14. A Transcranial Magnetic Stimulation system, the system comprising:
a TMS electromagnet;
a perturbing actuator connected to the TMS electromagnet comprising a voice coil, wherein the perturbing actuator is configured to mechanically oscillate the TMS electromagnet at a frequency of between about 1 kHz and 10 kHz; and
a controller configured to trigger activation of the TMS electromagnet and mechanical oscillation of the TMS electromagnet.

15. A method of applying Transcranial Magnetic Stimulation (TMS) to a subject, the method comprising:
positioning a magnet toward a target brain region;
emitting a magnetic field from the magnet towards the target tissue; and
mechanically oscillating the magnet at a frequency of greater than 1 KHz so that magnetic field emitted by the magnet moves relative to the target tissue and electrically perturbs the target tissue.

16. The method of claim 15, further comprising synchronously emitting the magnetic field and mechanically oscillating the magnet.

17. The method of claim 15, wherein the step of positioning the magnet comprises fixing the position of the magnet relative to a subject's head.

18. The method of claim 15, wherein the step of mechanically oscillating the magnet comprises rotating the magnet on its central axis.

19. The method of claim 15, wherein the step of mechanically oscillating the magnet comprises moving the magnet in and out on its central axis.

20. The method of claim 15, wherein the step of mechanically oscillating the magnet comprises moving the magnet laterally on and off the central axis.

21. The method of claim 15, wherein the step of mechanically oscillating the magnet comprises moving the magnet laterally partially on and off the central axis by moving the magnet about a fixed point on the central axis.

22. The method of claim 15, wherein the step of mechanically oscillating the magnet comprises oscillating the magnet at a frequency of between about 2 KHz and about 10 kHz.

23. The device of claim 1, wherein the magnet is a TMS electromagnet.

24. The device of claim 1, wherein the magnet is a permanent magnet.

25. The method of claim 15, wherein the step of positioning a magnet comprises positioning a TMS electromagnet.

26. The method of claim 15, where in the step of positioning a magnet comprises positioning a permanent magnet.
